SEQUENCE LISTING (6 sequences)

SEQ ID NO: 1 - protein, 14 aa, synthetic construct
  source 1..14: note = Adenovirus type 5
TPAPSPNCRLNAEK

SEQ ID NO: 2 - protein, 14 aa, synthetic construct
  source 1..14: note = Adenovirus type 5
TPAPSPNVTINRSA

SEQ ID NO: 3 - protein, 14 aa, synthetic construct
  source 1..14: note = Adenovirus type 5
TPAPSPNTYMLSRN

SEQ ID NO: 4 - protein, 14 aa, synthetic construct
TPAPSPNSTMGTSH

SEQ ID NO: 5 - DNA, 18 bp, synthetic construct
  source 1..18: mol_type = other DNA; note = primer
ggagtgcgccgagacaac

SEQ ID NO: 6 - protein, 19 aa, synthetic construct
  source 1..19: note = Primer
ACTACGTCCGGCGTTCCAT

(19) United States
(12) Patent Application Publication
(10) Pub. No.: US 2025/0249055 A1
(43) Pub. Date: Aug. 7, 2025

(54) ADENOVIRUS CONSTRUCTS AND METHODS OF USE

(71) Applicant: REGENTS OF THE UNIVERSITY OF MINNESOTA, Minneapolis, MN (US)

(72) Inventors: Masato Yamamoto, Golden Valley, MN (US); Mizuho Dahlman, Elk River, MN (US)

(21) Appl. No.: 18/854,172

(22) PCT Filed: Apr. 7, 2023
(86) PCT No.: PCT/US2023/017811

§ 371 Date: Oct. 4, 2024

Related U.S. Application Data
(60) Provisional application No. 63/328,372, filed on Apr. 7, 2022.

(51) Int. Cl. A61K 35/761; A61K 48/00; C07K 14/005; C12N 7/00; C12N 15/86

(52) U.S. Cl. CPC A61K 35/761; A61K 48/0041; C07K 14/005; C12N 7/00; C12N 15/86; C12N 2710/10021; C12N 2710/10022; C12N 2710/10032; C12N 2710/10043

(57) ABSTRACT

An adenovirus that reduces the extent to which neutralizing antibodies bind to the adenovirus has an AB loop that includes a sufficient portion of the amino acid sequence VTINRSA (amino acids 8-14 of SEQ ID NO:2), TYMLSRN (amino acids 8-14 of SEQ ID NO:3), or STMGTSH motif (amino acids 8-14 of SEQ ID NO:4) to reduce binding of neutralizing anti-adenovirus antibodies compared to an adenovirus having a wild-type AB loop. In some cases, another aspect, the adenovirus has an AB loop that includes a sufficient portion of the amino acid sequence VTINRSA (amino acids 8-14 of SEQ ID NO:2), TYMLSRN (amino acids 8-14 of SEQ ID NO:3), or STMGTSH motif (amino acids 8-14 of SEQ ID NO:4) to reduce hemagglutination compared to an adenovirus having a wild-type AB loop. The adenovirus can be used to deliver therapy to a target cell to which the AB loop binds.

### CROSS-REFERENCE TO RELATED APPLICATION

This application claims the benefit of U.S. Provisional Patent Application No. 63/328,372, filed Apr. 7, 2022, which is incorporated herein by reference in its entirety

### GOVERNMENT FUNDING

This invention was made with government support under CA196215 and CA168448 awarded by the National Institutes of Health. The government has certain rights in the invention.

### SUMMARY

This disclosure describes, in one aspect, an adenovirus having an AB loop that includes a sufficient portion of a variant of the wild-type AB loop amino acid sequence to reduce binding of neutralizing anti-adenovirus antibodies compared to an adenovirus having a wild-type AB loop.

In another aspect, this disclosure describes an adenovirus having an AB loop that includes a sufficient portion of a variant of the wild-type AB loop amino acid sequence to reduce hemagglutination compared to an adenovirus having a wild-type AB loop.

In either preceding aspect, the variant of the AB loop can include the amino acid sequence VTINRSA (amino acids 8-14 of SEQ ID NO:2), TYMLSRN (amino acids 8-14 of SEQ ID NO:3), or STMGTSH (amino acids 8-14 of SEQ ID NO:4).

In either preceding aspect, the adenovirus can further include a polynucleotide sequence that encodes a therapeutic agent. In one or more embodiments, the therapeutic agent includes an anti-tumor therapeutic agent.

In another aspect, this disclosure describes a composition that includes any embodiment of the adenovirus summarized above and a pharmaceutically acceptable carrier.

In another aspect, this disclosure describes a method of delivering therapy to a subject. Generally, the method includes administering to the subject any embodiment of the adenovirus of summarized above, allowing the adenovirus to bind to a target cell that expresses a marker recognized by the adenovirus, and allowing the adenovirus to infect the target cell, thereby delivering the therapy.

In one or more embodiments, the therapy includes allowing the virus to replicate in the target cell and lysis of the target cell.

In one or more embodiments, the therapy includes delivery of a therapeutic polynucleotide to the target cell and allowing the target cell to express the therapeutic polynucleotide.

In one or more embodiments, the target call is a tumor cell and the therapy is an anti-tumor therapy. In one or more of these embodiments, the tumor cell expresses mesothelin. In one or more alternative embodiments, the tumor cell expresses CD133. In one or more alternative embodiments, the tumor cell expresses prostate specific membrane antigen (PMSA).

In one or more embodiments, the adenovirus is administered systemically.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Generation of oncolytic adenovirus (OAd) with a modified AB loop region. (A) Structure of adenoviruses. (B) AB loop sequences of adenovirus constructs. Wild-type AB loop of fiber knob (SEQ ID NO:1; top), VTINRSA motif in AB loop of evolved mesothelin-binding adenovirus (SEQ ID NO:2; middle), and TYMLSRN motif in AB loop of evolved CD133-binding adenovirus (SEQ ID NO:3; bottom)

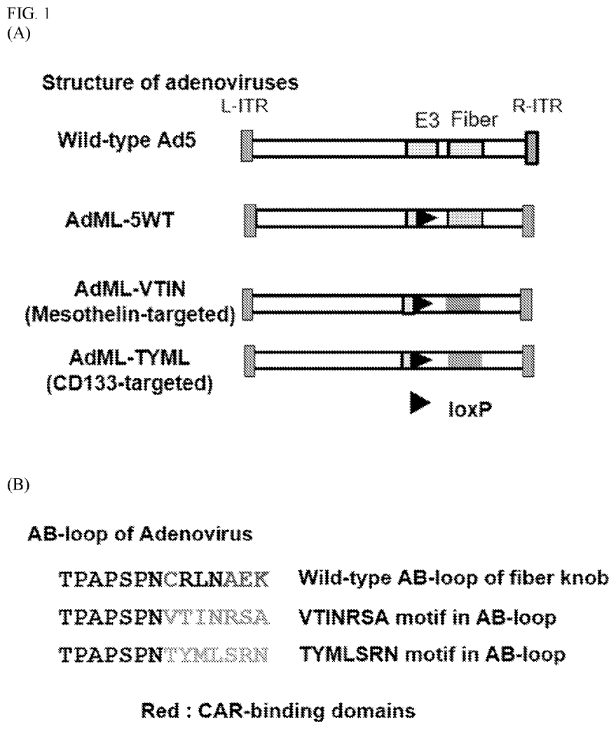

FIG. 2. Mesothelin-targeted OAd reduces neutralization by anti-adenovirus antibody. A549 (human lung carcinoma cells) cells were seeded in to 96-well plates at 5×10⁴ cells in 100 μl of culture media. On the following day, human serum was heat inactivated at 56° C. for 60 minutes before a serial dilution was performed in a 96-well tissue culture plate. The serum was serially diluted and mixed with 100 TCID₅₀ of Ads (Ad5-WT or AdML-VTIN). After a 30-minute incubation at 37° C., A549 cells were infected with the virus/serum mixture. After a seven-day incubation, the number of wells that are positive for CPE were scored and the CPE positivity was calculated.

FIG. 3. Neutralizing activity of individual human serums against Ad-5WT or AdML-VTIN. (A) Neutralizing activity of 1:4 dilution of human sera samples and 1:40 dilution of human serum sample #771. (B) Table of serum dilution of 50% virus killing inhibition. The dilution factor of 50% virus killing inhibition was determined from FIG. 2(A). 1:4 dilution of human serum is the highest concentration in this assay. Thus, 50% inhibition of less than 1:4 dilution is interpreted as no inhibition.

FIG. 4. CD133-targeted oncolytic adenovirus (AdML-TYML) escapes from anti-adenovirus antibody in human serum. HEK293 cells overexpressing CD133 (CD133-293 cells) were seeded in to 96-well plates at 5×10⁴ cells in 100 μl of culture media. On the following day, human serum was heat inactivated at 56° C. for 60 minutes before a serial dilution was performed in a 96-well tissue culture plate. The serum was serially diluted, and mixed with 500 vp/cell (10 pfu/cell) of Ads (AdML-5WT, or AdML-TYML). After a 30-minute incubation at 37° C., CD133-293 cells were infected with the virus/serum mixture. After a 14-day incubation, the number of wells that are positive for CPE were scored and the CPE positivity was calculated.

FIG. 5. Hemagglutination profiling of AB loop modified oncolytic adenoviruses. Ad5-based adenoviruses with CAR-binding knob showed hemagglutination (Ad5, and Ad5-RGD). Replacement of the knobs with AB loops that bind to CD133 (AdML-TYML), MSLN (AdML-VTIN), or CD46/DSG-2 (Ad5/3) did not cause hemagglutination.

### DETAILED DESCRIPTION OF ILLUSTRATIVE EMBODIMENTS

This disclosure describes adenovirus constructs that contain modifications to the AB loop of the fiber knob that reduce the adenovirus susceptibility to naturally-occurring anti-adenovirus neutralizing antibodies. This disclosure further describes methods of using the adenovirus constructs to deliver therapy to a target cell.

Oncolytic virotherapy is a promising anti-cancer platform. For example, adenovirus-based vectors used in gene therapy clinical trials. However, one challenge for systemic treatment with oncolytic adenovirus is the rapid neutralization of viral particles by pre-existing anti-adenovirus neutralizing antibodies (nAb). Particularly, human adenovirus serotype 5 shows high prevalence of virus-neutralizing antibodies in the human population.

Anti-adenovirus neutralizing antibodies mainly recognize three major capsid proteins: the hexon, fiber, and penton base proteins. One current strategy for escaping anti-adenovirus neutralizing antibody is switching the backbone vector or capsid proteins to other serotypes of adenovirus. Backbone switch strategy involves using rare serotypes of adenovirus—e.g., human adenovirus 26, 48, or 35—for cancer treatment or vaccine vectors. The Ad26 vector platform has been used as a vaccine vectors because Ad26 neutralizing antibody titers remain markedly lower than Ad5 neutralizing antibody titers. Another strategy is hexon gene switch strategy. Chimeric recombinant Ad5 vectors (e.g., Ad5HVR48) in which the short hypervariable regions (HVRs) on the surface of the Ad5 hexon protein are replaced with the corresponding HVRs from a rare adenovirus serotype (e.g., Ad48) can evade pre-existing Ad5 immunity. Moreover, a chimeric adenoviral vector (Ad5.F35) derived from the capsid of Ad5 and fiber of the rare adenovirus serotype 35 (Ad35) resisted neutralization in sera collected from patients with colorectal cancer naturally exposed to Ad5. Although serotype-switch strategies have been reported to circumvent pre-existing neutralizing antibodies, the success rate of Ad-capsid modification has been generally low. Further capsid modifications can affect viral capsid assembly, replication, and/or the vector yield.

This disclosure describes fiber-modified oncolytic adenoviruses (OAds) that are able to escape from pre-existing anti-adenovirus neutralizing antibodies and avoid hemagglutination. In a neutralizing antibody assay, the fiber-modified OAds (AdML-TYML, AdML-VTIN, AdML-STMG, etc.) were more resistant to human serum compared to fiber-unmodified OAd (Ad-wt). These modifications do not reduce transduction efficiency. These data suggest that oncolytic adenovirus constructs having modifications in the AB loop allow a degree of escape from pre-existing anti-adenovirus neutralizing antibodies and therefore have a greater potential to provide therapeutic vector for systemic therapy. Because the fiber-modified adenovirus vectors described herein are generated by using the replication-based Ad library screening system, the fiber-modified Ad vectors described herein do not affect viral production.

Methods for generating adenovirus libraries with targeting motifs in the AB loop are described in, for example, U.S. Pat. Nos. 10,208,304; 11,162,092; and U.S. Patent Publication No. US 2022/0042006 A1. The methods allowed identification of mesothelin-targeted oncolytic adenoviruses. Mesothelin is a cell surface glycoprotein that is highly expressed on pancreatic cancer tumor cells, ovarian cancer tumor cells, and mesothelioma cells with little to ne expression on cells in normal, healthy tissues. Mesothelin-targeted oncolytic adenoviruses showed selectivity for MSLN-expressing cells in vitro. In vivo, it exhibited selective and potent antitumor effects.

This disclosure presents data that show that modifications in the AB loop can further provide the adenovirus with the ability to escape naturally-occurring anti-adenovirus neutralizing antibodies, and avoid hemagglutination. Thus, in certain embodiments, modifications within the AB loop can provide dual activity of targeting the adenovirus to a particular target cell population and reduce the likelihood and/or extent to which neutralizing antibodies interfere with the adenovirus reaching and transducing the target cell.

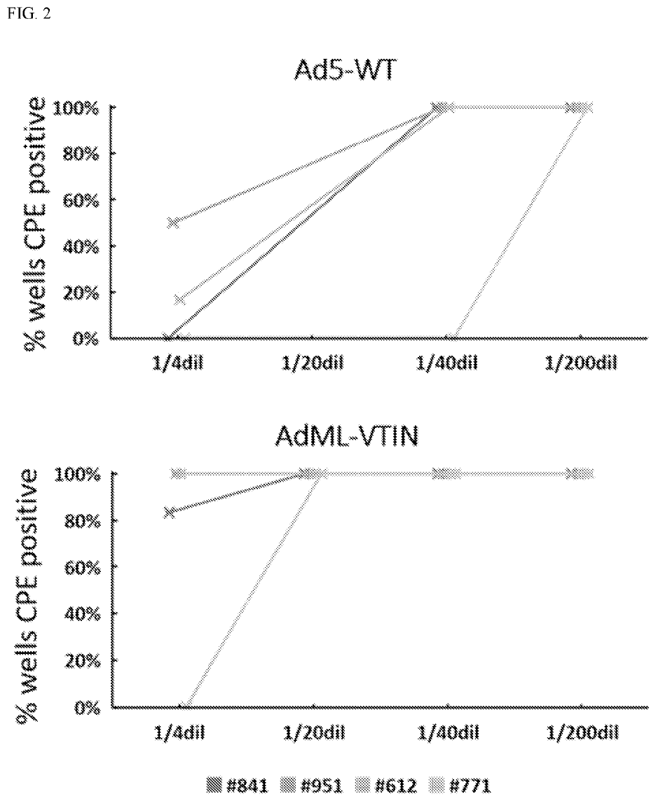

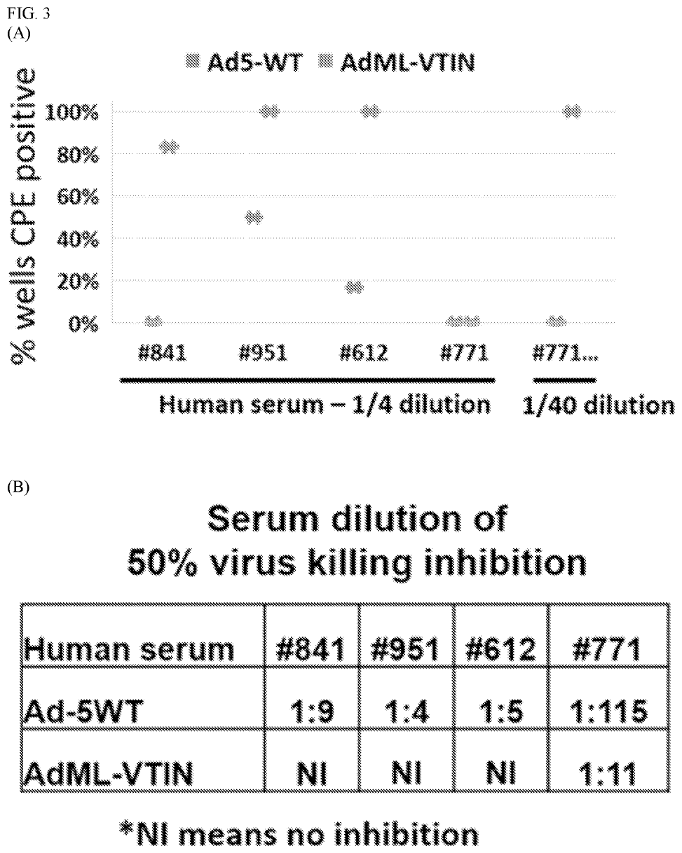

The ability of Ad5 and AdML-VTIN to escape neutralization by neutralizing antibodies (NAb) was evaluated using human serum from four healthy adults (FIG. 2 and FIG. 3). In a 1:4 dilution of serum condition, less than 50% of the wells showed cytopathic effect (CPE) in Ad5-WT infected cells, indicating that more than 50% of the Ad5-WT virus was neutralized by neutralizing antibodies (FIG. 3A). In contrast, AdML-VTIN virus killed more than 80% of the cells at the same serum dilution condition, with one exception: serum sample #771 (FIG. 2 and FIG. 3A). However, the AdTML-VTIN virus showed complete escape from neutralizing antibodies (100% CPE) in serum sample #771 at a 1:40 dilution of the serum sample (FIG. 3A). In addition, when the serum dilution of 50% virus killing inhibition was calculated, three out of four sera showed no inhibition of AdML-VTIN under a 1:4 dilution condition (FIG. 3B). The 1:4 dilution condition is a detection limit for this assay, indicating that dilution of less 1:4 dilution was regarded as no inhibition—i.e., AdML-VTIN displayed escape from neutralizing antibodies present in the serum samples.

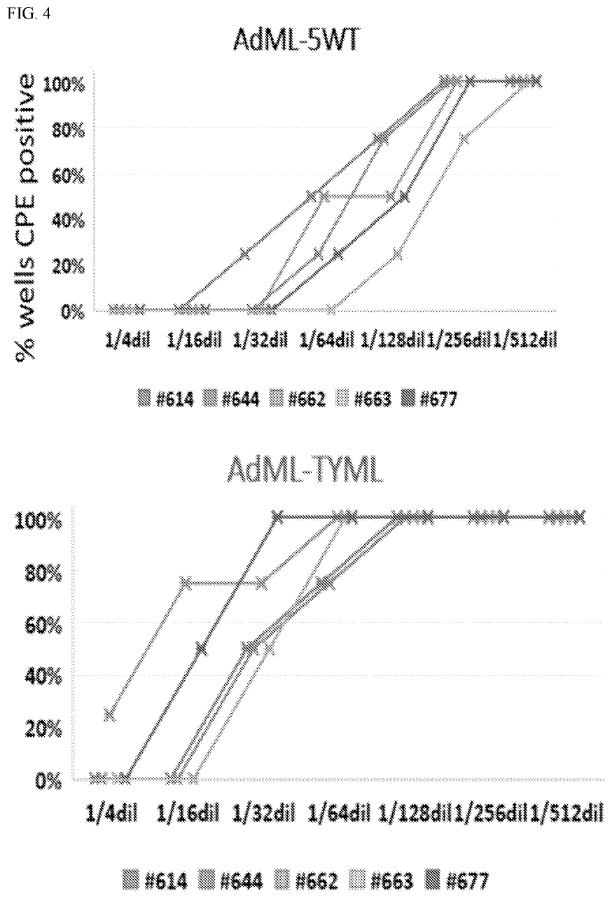

To evaluate the ability of the AB loop modified OAd to escape neutralizing antibodies, neutralizing antibody (NAb) responses were tested against another AB loop modified OAd, AdML-TYML, in human serum from five healthy adults (FIG. 4). AdML-TYML produced more than 70% CPE-positive wells at 1:64 serum dilution condition but AdML-5WT virus produced less than 50% CPE-positive wells at the same condition, indicating that AdML-TYML was more resistant to neutralizing antibodies in human serum compared to fiber-unmodified OAd (AdML-5WT) (FIG. 4). These data suggest that OAd with a targeting motif in the AB loop allows a degree of escape from pre-existing Ab.

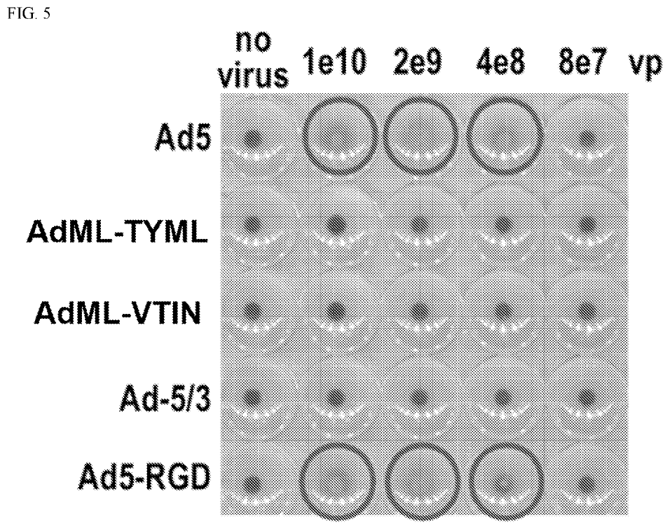

In addition, the hemagglutination activity of AB loop modified OAds (AdML-VTIN and AdML-TYML) was evaluated because binding of the Ad fiber-knob protein to erythrocytes promotes hemagglutination. Ad5-based Ads with a CAR-binding knob showed hemagglutination (Ad5, and Ad5-RGD). In contrast, Ads with a modified AB loop (Ad5-TYML and Ad5-VTIN) and virus having an Ad3 fiber-knob rather than the Ad5 fiber-knob (Ad5/3) did not cause hemagglutination (FIG. 5). These data show that AB loop modification is an effective way to avoid hemagglutination.

Thus, this disclosure describes target-specific Ad5-based adenoviruses in which target-specific variations in the AB loop (e.g., in the CAR-binding domain) of the fiber knob confer the ability of the variant Ad5-based adenovirus to escape neutralizing antibodies present in human serum to a greater degree than Ad5-based adenoviruses having a wild-type AB loop (SEQ ID NO: 1). This disclosure provides data demonstrating escape for neutralizing antibodies exhibited by variant Ad5-based adenoviruses having a VTINRSA motif (amino acids 8-14 of SEQ ID NO: 2) or a TYMLSRN motif (amino acids 8-14 of SEQ ID NO:3) replacing the CAR-binding domain of the AB loop. This disclosure further describes an additional Ad5-based adenovirus variant that has a STMGTSH motif (amino acids 8-14 of SEQ ID NO:4) replacing the CAR-binding domain of the AB loop. Modifications to the AB loop, particularly in the CAR-binding domain, that modify the targeting of Ad5-based adenovirus interfere with recognition of the variant Ad5-based adenovirus by neutralizing antibodies. Thus, this disclosure describes a general platform that allows one to design Ad5-based adenoviruses modified for selective targeting and escape from neutralizing antibodies.

In another aspect, this disclosure describes methods of delivering therapy using the variant Ad5-based adenoviruses described herein. Generally, the methods include administering a target-specific variant Ad5-based adenovirus, as described above, allowing the adenovirus to bind to a target cell that expresses a marker recognized by the adenovirus, and allowing the adenovirus to infect the target cell, thereby delivering the therapy. As described in more detail above, the target specificity that allows the adenovirus to recognize the marker expressed by the target cell may further confer the ability of the variant Ad5-based adenovirus to escape neutralizing antibodies present in the serum of the subject, thereby increasing the likelihood and/or extent to which the variant adenovirus is delivered to the target cell.

In one or more embodiments, the therapy can include replication of the adenovirus inside the target cell and lysis of the target cell. In one or more alternative embodiments, the therapy can include delivery of a therapeutic polynucleotide and allowing the target cell to express the therapeutic polynucleotide. The therapeutic polynucleotide may encode a therapeutic polypeptide, may increase expression of an endogenous polynucleotide or an endogenous polypeptide, or may inhibit expression of an endogenous polynucleotide or an endogenous polypeptide.

In one or more embodiments, the target cell is a tumor cell and the therapy is an anti-tumor therapy. In one or more of these embodiments, the tumor cell may express and the variant Ad5-based adenovirus may recognize and bind to, mesothelin. In one or more alternative embodiments, the tumor cell may express and the variant Ad5-based adenovirus may recognize and bind to, CD133. In one or more alternative embodiments, the tumor cell may express and the variant Ad5-based adenovirus may recognize and bind to, prostate specific membrane antigen (PMSA).

In one or more embodiments, the target-specificity conferred by the variant AB loop allows systemic delivery while limiting off target effects that may be undesirable. The modifications to the AB loop that also confer escape from neutralizing antibodies increases the half-life of the modified Ad5-based adenovirus and increases the likelihood and/or extent to which the variant Ad5-based adenovirus reaches the target cell to deliver therapy.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an.” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “some embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Adenovirus Design

AdML-VTIN has a wild-type E1 gene, a single loxP site replicating the E3 gene, and VTINRSA motif replacing the primary CAR-binding domain in AB loop of the fiber knob.

AdML-TYML has a wild-type E1 gene, a single loxP site replicating the E3 gene, and TYMLSRN motif replacing the primary CAR-binding domain in AB loop of the fiber knob.

AdML-STMG has a wild-type E1 gene, a single loxP site replicating the E3 gene, and TYMLSRN motif replacing the primary CAR-binding domain in AB loop of the fiber knob.

Ad5-WT, control adenovirus, has a wild-type E1 gene, single loxP site replicating the E3 gene, and wild-type fiber of Ad5 (Miura et al., 2013, Mol Ther 21:139-148). The titer of the viruses was determined by optical absorbance at 260 nm, qPCR, and plaque assay as previously described (Yamamoto et al., 2003, Gastroenterology 125:1203-1218).

### Neutralization Assay

For neutralization assay with the mesothelin-targeted OAd, A549 (human lung carcinoma cells) cells were seeded in to 96-well plates at 5×10⁴ cells in 100 μl of culture media. On the following day, human serum was heat inactivated at 56° C. for 60 min before a serial dilution was performed in a 96-well tissue culture plate. The serum was serially diluted, and mixed with 100 TCID₅₀ of Ads (Ad5-WT, or AdML-VTIN). After a 30-minute incubation at 37° C., A549 cells were infected with the virus/serum mixture. After a seven-day incubation, the number of wells that are positive for CPE (cytopathic effect) were scored and the CPE positivity was calculated.

For neutralization assay with the CD133-targeted OAd, HEK293 cells overexpressing CD133 (CD133-293 cells) were seeded in to 96-well plates at 5×10⁴ cells in 100 μl of culture media. On the following day, human serum was heat inactivated at 56° C. for 60 minutes before a serial dilution was performed in a 96-well tissue culture plate. The serum was serially diluted, and mixed with 100 vp/cell of Ads (Ad5-WT, or AdML-TYML). After a 30-minute incubation at 37° C., CD133-293 cells were infected with the virus/serum mixture. After a 14-day incubation, the number of wells that are positive for CPE were scored and the CPE positivity was calculated.

### Hemagglutination Assay

Erythrocytes were extracted from blood cells derived from a human donor, who gave informed consent. Then, 50 μl of 0.5% (v/v) erythrocyte suspension was layered in each well of concave-bottom-shaped 96-well plate, and 50 μl of virus dilutions (1×10¹⁰ vp to 2.56×10⁴ vp in PBS) was added and gently mixing into the erythrocyte suspension. The plates were incubated 37° C. for two hours to allow sedimentation to occur. Hemagglutination was assessed visually.

### Quantitative Analysis for the Adenoviral Copy Number Determination

Viral solutions were treated with 0.1 U/μl of DNasel at 37° C. for 15 minutes for eliminating non-capsidated viral DNA. The DNA was purified with QIAamp Blood Kit (Qiagen, Hilden, Germany) following the manufacture's instruction. The total viral copy number was analyzed with the E4 primers by SYBRGreen quantitative PCR (qPCR) using QuantiFast SYBR Green PCR Kit (Qiagen, Hilden, Germany). Oligonucleotide sequences were, E4-forward: 5′-GGAGTGCGCCGAGACAAC-3′ (SEQ ID NO:5); E4-reverse: 5′-ACTACGTCCGGCGTTCCAT-3′ (SEQ ID NO:6).

### Statistical Analysis

Statistical comparisons between two groups were evaluated by Student's t-test. All P-values were two-sided, and a value of P<0.05 and P<0.01 was considered to indicate statistical significance.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1-Wild-type AB loop sequence
TPAPSPNCRL NAEK
SEQ ID NO: 2-VTINRSA AB loop sequence
TPAPSPNVTI NRSA
SEQ ID NO: 3-TYML AB loop sequence
TPAPSPNTYM LSRN
SEQ ID NO: 4-STMG AB loop sequence
TPAPSPNSTM GTSH
SEQ ID NO: 5-E4-forward:
GGAGTGCGCC GAGACAAC
SEQ ID NO: 6-E4-reverse:
ACTACGTCCG GCGTTCCAT

1. An adenovirus comprising an AB loop comprising a sufficient portion of a variant AB loop amino acid sequence to reduce binding of neutralizing anti-adenovirus antibodies compared to an adenovirus having a wild-type AB loop.

2. An adenovirus comprising an AB loop comprising a sufficient portion of a variant AB loop amino acid sequence to reduce hemagglutination compared to an adenovirus having a wild-type AB loop.

3. The adenovirus of claim 1, wherein the variant AB loop amino acid sequence comprises at least one amino acid substitution.

4. The adenovirus of claim 1, wherein the amino acid substitution is in the CAR-binding domain of the AB loop.

5. The adenovirus of claim 1, wherein the variant AB loop amino acid sequence comprises:
amino acids 8-14 of SEQ ID NO:2,
amino acids 8-14 of SEQ ID NO:3, or
amino acids 8-14 of SEQ ID NO:4.

6. The adenovirus of claim 1, further comprising a polynucleotide sequence that encodes a therapeutic agent.

7. The adenovirus of claim 6, wherein the therapeutic agent comprises an anti-tumor therapeutic agent.

8. A composition comprising:
the adenovirus of claim 1; and
a pharmaceutically acceptable carrier.

9. A method of delivering a therapy to a subject, the method comprising:
administering to the subject the adenovirus of claim 1;
allowing the adenovirus to bind to a target cell that expresses a marker recognized by the adenovirus; and
allowing the adenovirus to infect the target cell, thereby delivering the therapy.

10. The method of claim 9, wherein the therapy comprises:
allowing the adenovirus to replicate in the target cell; and
lysis of the target cell.

11. The method of claim 9, wherein the therapy comprises:
delivery of a therapeutic polynucleotide; and
allowing the target cell to express the therapeutic polynucleotide.

12. The method of claim 9, wherein the target cell is a tumor cell and the therapy is an anti-tumor therapy.

13. The method of claim 11, wherein the tumor cell expresses mesothelin.

14. The method of claim 11, wherein the tumor cell expresses CD133.

15. The method of claim 11, wherein the tumor cell expresses prostate specific membrane antigen (PMSA).

16. The method of claim 9, wherein the adenovirus is administered systemically.
